(19) United States
(12) Patent Application Publication
(10) Pub. No.: US 2006/0106014 A1
(43) Pub. Date: May 18, 2006

(54) Methods for treating diabetes

(76) Inventors: Sekhar Boddupalli, San Jose, CA (US); David N. Liebowitz, San Jose, CA (US); Guy Miller, San Jose, CA (US)

Correspondence Address: FOLEY & LARDNER LLP, 1530 PAGE MILL ROAD, PALO ALTO, CA, 94304, US

(21) Appl. No.: 11/251,423

(22) Filed: Oct. 13, 2005

Related U.S. Application Data
(60) Provisional application No. 60/619,085, filed on Oct. 14, 2004.

(51) Int. Cl. A61K 31/5377; A61K 31/52; A61K 31/506; A61K 31/497; A61K 31/501; A61K 31/452; A61K 31/473; A61K 31/407; A61K 31/404; A61K 31/4015; A61K 31/353

(52) U.S. Cl. 514/232.5; 514/252.01; 514/254.11; 514/255.05; 514/314; 514/263.24; 514/305; 514/320; 514/337; 514/248; 514/297; 514/414; 514/365; 514/372; 514/374; 514/411; 514/256; 514/422; 514/456; 514/406; 514/397

(57) ABSTRACT

Disclosed are methods for the treatment of diabetes by administering a composition comprising a pharmaceutically acceptable amount of a dual inhibitor of both 5-lipoxygenase (5-LO) and 12/15-lipoxygenase (12/15-LO) enzymes or of both 5-lipoxygenase (5-LO) and 15-lipoxygenase (15-LO) enzymes. The invention is also directed to methods of controlling blood glucose level in diabetic patients with a dual inhibitor of both 5-lipoxygenase (5-LO) and 12/15-lipoxygenase (12/15-LO) enzymes or of both 5-lipoxygenase (5-LO) and 15-lipoxygenase (15-LO) enzymes.

### CROSS-REFERENCE TO RELATED APPLICATIONS

This application claims the benefit under 35 U.S.C. 119(e) of U.S. Provisional Application Ser. No. 60/619,085, filed Oct. 14, 2004, incorporated herein by reference in its entirety.

### FIELD OF THE INVENTION

The present invention is concerned with a method for enhancing glucose control in a subject in need of such control which comprises administering to the subject a composition comprising a therapeutically effective amount of one or more dual inhibitors of any two lipoxygenase enzymes, particularly of both 5-lipoxygenase (5-LO) and 12/15-lipoxygenase (12/15-LO) enzymes, or of both 5-lipoxygenase (5-LO) and 15-lipoxygenase (15-LO) enzymes. The methods of this invention are particularly applicable to preventing and/or treating diabetes in a subject in need thereof.

### BACKGROUND INFORMATION

Lipoxygenases are nonheme iron-containing enzymes found in plants and animals that catalyze the oxygenation of certain polyunsaturated fatty acids, such as lipids and lipoproteins. Several different lipoxygenase enzymes are known, each having a characteristic oxidation action. Mammalian lipoxygenases are named by the position in arachidonic acid that is oxygenated. The enzyme 5-lipoxygenase converts arachidonic acid to 5-hydroperoxy-eicosatetraenoic acid (5-HPETE). This is the first step in the metabolic pathway which yields 5-hydroxyeicosatetraenoic acid (5-HETE) and the leukotrienes (LTs). Similarly, 12- and 15-lipoxygenase convert arachidonic acid to 12- and 15-HPETE, respectively. Biochemical reduction of 12-HPETE leads to 12-HETE, while 15-HETE is the precursor of the class of compounds known as lipoxins. A diverse array of biological effects are associated with the products of lipoxygenase activity, and many are implicated as mediators in various disease states. The C₄ and D₄ leukotrienes are potent constrictors of human bronchial smooth muscle; LTB₄ and 5-HETE, found in the synovial fluid of patients with rheumatoid arthritis, are potent chemotactic factors for inflammatory cells such as polymorphonuclear leukocytes (Green and Lambeth. Tetrahedron, Vol. 39 (1983), pp. 1687); 12-HETE has been found at high levels in the epidermal tissue of patients with psoriasis; the lipoxins have been shown to stimulate liposomal enzyme and superoxide ion release from neutrophils. Thus, lipoxygenase enzymes play an important role in the biosynthesis of mediators of asthma, allergy, arthritis, psoriasis, and inflammation, and inhibitors of these enzymes interrupt the biochemical pathway involved in these disease states.

Human 15-lipoxygenase (15-LO) catalyzes the formation of 15-S-hydroxyeicosatetraenoic acid (15-S-HETE) from arachidonic acid (Kuhn and Borngraber. Lipoxygenases and Their Metabolites. New York, Plenum Press, 1999). In mice, the synthesis of 15-S-HETE is carried out by 12/15-Lipoxygenase which is the murine homologue of human 15-LO. Murine 12/15-LO converts arachidonic acid to 12(S)-hydroxyeicosatetraenoic and 15-S-HETE in a 3:1 ratio, and can additionally convert linoleic acid to 13-hydroxyoctadecadienoic acid (13-HODE).

15-Lipoxygenase has previously been implicated in the pathogenesis of several diseases, including atherosclerosis (Harats et al. Arterioscler. Thromb. Vasc. Biol., 2000, pp. 2100-2105), asthma (Shannon et al. Am. Rev. Respir. Dis., Vol. 147 (1993), pp. 1024-1028), cancer (Shureiqi et al. JNCI, Vol. 92 (2000), pp. 1136-1142), and glomerulonephritis (Montero and Bard. Exp. Neph., Vol. 8 (2000), pp. 14-19). A number of classes of compounds have been identified that inhibit 15-LO, including phenols, hydroxamic acids and acetylenic fatty acids (reviewed in Kuhn and Borngraber. Lipoxygenases and Their Metabolites. New York, Plenum Press, 1999). The spectrum of inhibitory activities varies for these agents. For example, nordihydroguaiaretic acid (NDGA) has been shown to be an inhibitor of 5- and 15-lipoxygenase, naphthyl hydroxamic acids have been shown to inhibit 5-, 12-, and 15-lipoxygenase (U.S. Pat. No. 4,605,669), a benzofluorene 15-LO inhibitor, PD146176, has been reported to be relatively specific for the 15-LO enzyme (Sendobry et al. Br. J. Pharm., Vol. 120 (1997), pp. 1199-1206), and a benzothiophene 5-LO inhibitor, A-64077, (Zileuton, U.S. Pat. No. 4,873,259) has been reported to be specific for the 5-LO enzyme (see e.g., Bell R. L. et al. Int. J. Immunopharmacol., Vol. 14, no. 3 (1992), pp. 505-10).

Although 5-LO and 12/15-LO have been implicated in the pathogenesis of several diseases, the human clinical utility for dual inhibitors of both 5-LO and 12/15-LO enzymes, or of both 5-LO and 15-LO enzymes for controlling glucose has not previously been discovered.

Diabetes is caused by occurrence of abnormal metabolisms of glucose, protein, and lipids due to a deficiency or insufficiency of the actions of insulin. Typical signs of diabetes include an abnormal increase in the serum glucose level over the normal range of the glucose level, and an excretion of glucose in the urine.

Several clinical subclasses are recognized, including: Type 1 (insulin-dependent or IDDM), Type 2 (non-insulin-dependent diabetes mellitus), maturity-onset diabetes of the young (MODY) and gestational diabetes. They differ in etiology, pathology, genetics, age of onset, and treatment.

Type 1 diabetes, the more severe form of diabetes, accounts for 5 to 10 percent of diabetes and occurs most often in children and young adults. In this form of diabetes the body does not produce any insulin. Without regular injections of insulin the sufferer lapses into a coma and dies. Individuals suffering from Type 1 diabetes are totally insulin dependent.

Type 2 diabetes, the more prevalent type of diabetes, is usually characterized by gradual onset and occurs mainly in people over 40. Type 2 diabetes is a metabolic disorder resulting from the body's inability to make enough insulin or to properly use insulin to meet the body's needs, especially when the person is overweight. It is the most common form of the disease. Type 2 diabetes accounts for 90 to 95 percent of diabetes. Type 2 diabetes is nearing epidemic proportions due to a greater prevalence of obesity and sedentary lifestyles. Initially, the combination of dietary measures, weight reduction and oral medication can keep the condition under control for a period of time, but most people with Type 2 diabetes ultimately require insulin injections.

Diabetes may be controlled with insulin and in some cases through careful diet, but there is a need for a safe and effective treatment for diabetes with minimal side effects and without the invasive procedure of insulin injection.

### SUMMARY OF THE INVENTION

The present invention is based on the discovery that dual inhibitors of any two lipoxygenase enzymes, particularly of 5- and 12/15-lipoxygenase enzymes or of 5- and 15-lipoxygenase enzymes, are able to improve glucose control in animal models of diabetes and have demonstrated a significant lowering of the baseline serum glucose levels compared to selective 5-LO, 15-LO and 12/15-LO inhibitors.

The present invention is concerned with a method of preventing or treating diabetes with a composition comprising:

a) identifying a subject susceptible to diabetes;

b) administering to the subject a composition comprising a pharmaceutically effective amount of one or more dual inhibitors of both 5-LO and 12/15-LO enzymes or of both 5-LO and 15-LO enzymes. In one embodiment the subject is susceptible to Type 1 diabetes, and in another embodiment the subject is susceptible to Type 2 diabetes, for example the subject is a mammal, such as a human.

In some embodiments, the dual inhibitor exhibits an in vitro IC₅₀ value of less than 5 micromolar in both a 5-LO enzyme assay and a 12/15-LO enzyme assay or in both a 5-LO enzyme assay and a 15-LO enzyme assay.

In another embodiment, the method of preventing or treating relates to controlling the blood glucose level in a subject with an elevated blood glucose level with a composition comprising a pharmaceutically effective amount of one or more dual inhibitors of both 5-LO and 12/15-LO enzymes or of both 5-LO and 15-LO enzymes, for example the subject is a mammal, such as a human. In another embodiment the method of preventing or treating relates to a subject susceptible of insulinitis, the method comprising administering to the subject a composition comprising a pharmaceutically effective amount of one or more dual inhibitors of both 5-LO and 12/15-LO enzymes or of both 5-LO and 12/15-LO enzymes, for example the subject is a mammal, such as a human.

In some embodiments, the dual inhibitor is selected from the group consisting of nordihydroguaiaretic acid (NDGA), 8-fluoro-2,2,5,7-tetramethylchroman-6-ol, and 1-(4-hydroxyphenyl)-2,7,8-trimethyl-1,2,3,4-tetrahydroquinolin-6-ol.

In several particular embodiments the dual inhibitor is represented by Formula I: 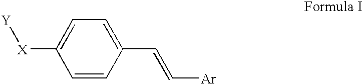
wherein
Ar is an aryl group optionally substituted with one or more groups independently selected from alkyl, alkenyl, hydroxy, alkoxy, carboxy, amido, sulfonyl, aminosulfonyl, cyano, nitro and halogen;
X is a bond, an alkylene or an alkenylene group, and
Y is nitro, cyano, carboxy, amino, sulfonylamino, aminosulfonyl, alkylsulfonyl, arylsulfonyl, heterocyclylsulfonyl, or heterocyclic selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
   single stereoisomers and mixtures of stereoisomers, prodrugs, and pharmaceutically acceptable salts thereof.

Some exemplary compounds of Formula I exhibiting an in vitro IC₅₀ value of less than 5 micromolar in both a 5-LO enzyme assay and a 12/15 enzyme assay, are:
4-[2-(4-bromo-phenyl)-vinyl]-benzene-1,2-diol;
4-[2-(4-tert-Butyl-phenyl)-vinyl]-1,2-bis-methoxymethoxy-benzene;
1,2-bis-methoxymethoxy-4-[2-(4-trifluoromethyl-phenyl)-vinyl]-benzene;
2,6-dimethyl-4-[2-(4-nitro-phenyl)-vinyl]-phenol;
4-{2-[3,4-dihydroxy-2-(3-methyl-but-2-enyl)-phenyl]-vinyl}benzoic acid methyl ester;
N-{4-[2-(3,4-dihydroxy-phenyl)-vinyl]-phenyl}acetamide;
N-{4-[2-(3,4-dihydroxy-phenyl)-vinyl]-phenyl}methanesulfonamide;
4-{2-[4-(piperidine-1-sulfonyl)-phenyl]-vinyl}benzene-1,2-diol;
4-[2-(2,3,4-trihydroxy-phenyl)-vinyl]-benzonitrile;
5-(4-nitrophenethyl)benzene-1,2,3-triol;
4-(3,4,5-trihydroxyphenethyl)benzonitrile;
5-(4-(methylamino)styryl)-3-(3-methylbut-2-enyl)benzene-1,2-diol;
5-(4-(dimethylamino)styryl)-3-(3-methylbut-2-enyl)benzene-1,2-diol;
4-(2,4-dihydroxystyryl)benzene-1,2-diol;
4-{2-[4-amino-sulfonyl)-phenyl]-vinyl}benzene-1,2-diol;
4-[2-(4-nitro-phenyl)-vinyl]-benzene-1,2-diol; and
4-[2-(3,4-dihydroxy-phenyl)-vinyl]-benzoic acid methyl ester.

It is also contemplated that these compounds will exhibit similar values in a 15-LO enzyme assay.

In other particular embodiments the dual inhibitor is represented by Formula II: 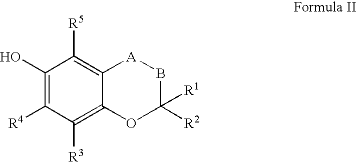
wherein,
R¹ is alkyl optionally substituted with halogen, hydroxy, cyano, amido or carboxy; or alkenyl optionally substituted with halogen, hydroxy, cyano, amido or carboxy;
R² is
   alkyl optionally substituted with halogen, hydroxy, cyano, or carboxy;
   alkenyl optionally substituted with halogen, hydroxy, cyano, or carboxy,
   aryl optionally substituted with one or more groups independently selected from alkyl, alkenyl, hydroxy, alkoxy, carboxy, amido, sulfonyl, aminosulfonyl, cyano, nitro and halogen; or
   heterocyclyl selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
R³ is hydrogen, optionally substituted alkyl, halogen, optionally substituted aryl or optionally substituted heterocyclyl;
R⁴, and R⁵ are independently of each other hydrogen, optionally substituted alkyl or halogen, or R⁴ and R⁵ may form an optionally substituted unsaturated or aromatic 5-6 membered ring optionally containing one or more heteroatoms or a C₇₋₁₂ bicyclo [a.b.c]alkyl ring where a, b, and c are 0 to 6, and
-A-B— is —CH₂—(CH₂)₀₋₂— or —CH═CH—; or
   single stereoisomers and mixtures of stereoisomers, prodrugs, and pharmaceutically acceptable salts thereof.

Some exemplary compounds of Formula II exhibiting an in vitro IC₅₀ value of less than 5 micromolar in both a 5-LO enzyme assay and a 12/15-LO enzyme assay, are:
5-allylsulfanylmethyl-2,2,7,8-tetramethyl-chroman-6-ol;
2-(2-chloro-ethyl)-2,7,8-trimethyl-chroman-6-ol;
7-bromo-2,2,5,8-tetramethyl-chroman-6-ol;
2,2,5,8-tetramethyl-7-(3-methyl-butyl)-chroman-6-ol;
2,2-dimethyl-3,4-dihydro-2H-benzo[h]chromene-6,10-diol;
8-fluoro-2,2,5,7-tetramethylchroman-6-ol;
8-iodo-2,2,5,7-tetramethylchroman-6-ol;
2,5,7,8-tetramethyl-2-(thiophen-2-yl)chroman-6-ol;
8-isopentyl-2,2,5,7-tetramethylchroman-6-ol;
5-methoxy-2,2,7,8-tetramethylchroman-6-ol;
2,2,5,7-tetramethyl-8-(trifluoromethyl)chroman-6-ol;
5-(furan-2-yl)-2,2,7,8-tetramethylchroman-6-ol;
2,5,7,8-tetramethyl-2-styrylchroman-6-ol;
5-chloro-2,2,7,8-tetramethylchroman-6-ol; and
2-(3,5-difluoro-4-hydroxyphenyl)-2,5,7,8-tetramethylchroman-6-ol.

It is also contemplated that these compounds will exhibit similar values in a 15-LO enzyme assay.

In some embodiments, the composition comprising a pharmaceutically effective amount of one or more dual inhibitors of both 5-LO and 12/15-LO enzymes or of both 5-LO and 15-LO enzymes is coadministered with another diabetic medication. In other embodiments, the composition comprises a pharmaceutically effective amount of one or more dual inhibitors of both 5-LO and 12/15-LO enzymes or of both 5-LO and 15-LO enzymes coadministered with another diabetic medication selected from the group consisting of pioglitizone, glimepiride, rosiglitazone, glipizide, metforministol, miglitol, repaglinide, acarbose, troglitazone, nateglinide, and combinations thereof.

Compounds useful in the practice of the present invention are not limited to compounds of Formula I and II, and it should be understood that other dual inhibitors of both 5-LO and 12/15-LO enzymes or of both 5-LO and 15-LO enzymes are also included in the invention provided that they exhibit adequate activity. The inhibitors may include synthetic organic molecules, plant extracts and other natural products.

### BRIEF DESCRIPTION OF DRAWINGS

FIG. 1 illustrates the IC₅₀'s of compounds A, B, C, D and E against 5-LO and 12/15-LO enzymes.

FIG. 2 illustrates the changes in the absolute levels of serum glucose. Compounds A, B, and C, at 30 mg/kg caused reduction in serum glucose relative to the respective vehicle control group with p<0.01, but compound A which is a dual inhibitor, showed a more pronounced effect. Similarly, compound E, a dual inhibitor, at 15 mg/kg caused significant lowering of serum glucose levels compared to its respective control with p<0.05. Compound D, a dual inhibitor, demonstrated 13% reduction in baseline elevated blood glucose level in comparison to the vehicle group. The data suggest that dual inhibition of both 5-LO and 12/15-LO results in superior efficacy compared to selective inhibition of either 5-LO or 12/15-LO. The dosages in mg/kg were chosen based on the pK properties of the compounds to achieve an IC₅₀ in vivo.

### DETAILED DESCRIPTION OF THE INVENTION

Definitions

As used in the present specification, the following words and phrases are generally intended to have the meanings as set forth below, except to the extent that the context in which they are used indicates otherwise.

“Diabetes” or diabetes mellitus is a metabolic disease that is defined by the presence of chronically elevated levels of blood glucose. Diabetes is caused by abnormal metabolism of glucose, protein and lipids, due to a deficiency or insufficiency of the actions of insulin. Typical signs of diabetes include an abnormal increase in the serum glucose level over the normal range of the glucose level and an excretion of glucose in the urine. Classic symptoms of diabetes mellitus in adults include polyuria, polydipsia, ketonuria, rapid weight loss, other acute manifestations of hyperglycemia, and elevated levels of plasma glucose.

Type 1 diabetes (also called insulin-dependent diabetes (IDDM), juvenile diabetes, brittle diabetes, or sugar diabetes) is accompanied by reduction of insulin producing cells, and Type 2 (also called non-insulin-dependent diabetes (NIDDM)) is caused by insulin sensitivity reduction or insulin secretion reduction. Symptoms of Type 1 diabetes include, but are not limited to, high levels of sugar in the blood when tested, high levels of sugar in the urine when tested, unusual thirst, frequent urination, extreme hunger but loss of body weight, blurred vision, nausea and vomiting, extreme weakness and tiredness, and irritability and mood changes. Complications associated with Type 1 include, but are not limited to, hypoglycemia (blood sugar drops too low, called insulin reaction), hyperglycemia (blood sugar is too high, indicating diabetes is not well controlled), and ketoacidosis (diabetic coma or loss of consciousness due to untreated or under-treated diabetes).

Type 2 diabetes is characterized by insulin resistance, i.e., a failure of the normal metabolic response of peripheral tissues to the action of insulin. Insulin resistance refers to a condition wherein the insulin level required to exhibit insulin activity at the same level as a healthy person is much higher than that of the healthy person. It is a condition wherein the activity of insulin or sensitivity for insulin is reduced. In clinical terms, insulin resistance is when normal or elevated blood glucose levels persist in the presence of normal or elevated levels of insulin. The hyperglycemia associated with Type 2 diabetes can be reversed or ameliorated by diet or weight loss sufficient to restore the sensitivity of the peripheral tissues to insulin. Progression of Type 2 diabetes includes increasing concentrations of blood glucose, coupled with a relative decrease in the rate of glucose-induced insulin secretion. Unlike the pancreatic beta cells in Type 1 diabetics, the beta cells of Type 2 diabetics retain the ability to synthesize and secrete insulin. Type 2 diabetes mellitus is often accompanied by obesity.

Type 2 also includes a non-insulin dependent diabetes mellitus of the young people, MODY (maturity-onset type of the diabetes in the young), and a morbid hyperglycemia caused by continuous administration of a steroid drug such as glucocorticoid (a steroid diabetes), or a hyperglycemia of Cushing Syndrome or an acromegaly because they are diabetes under normal or high level of insulin conditions. Diabetes mellitus also includes other specific types of diabetes mellitus and gestational diabetes mellitus.

There is also Type 3 and Type 4 diabetes. Type 3 diabetes typically refers to diabetes that is due to genetic defect in beta cells, genetically related insulin resistance, diseases of the pancreas, hormonal defects, or induced by chemicals or drugs. Type 4 diabetes refers to gestational diabetes that occurs in 2% to 5% of pregnancies.

“Insulinitis” refers to infiltration of lymphocytes into pancreatic islets of Langerhans and destruction of beta cells. Insulinitis is the most prominent histopathologic lesions in NOD mice (Makino, S. et al. Exp. Animal, Vol. 29 (1980), pp. 1-13). The first signs of pancreatic perivasculitis are seen at an age of about 15 days (Sugihara, T. et al. Histol. Histopathol., Vol. 4 (1989), pp. 397404, Miyazaki, A. et al. Clin. Exp. Immuno., Vol. 60 (1985), pp. 622-630). Lymphocyte infiltration is also observed in other organs such as salivary glands, thyroid glands, adrenal glands, testes, and ovaries. Upon complete onset of Type 1 diabetes, all of the pancreatic beta cells have been destroyed.

“Subject” refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” refers to a subject in need of treatment of a condition, disorder or disease, e.g., diabetes.

What is meant by “susceptible to diabetes” is that a subject has diabetes or is at risk for diabetes.

The susceptibility of a subject may be diagnosed, tested or identified by screening the patient. The screening may occur in a variety of ways but typically the screening involves measuring glucose levels in a patient. Glucose levels are expressed in milligrams of glucose per deciliter of blood (mg/dl) or millimoles per liter (mmol/L).

One diagnostic test is the fasting glucose test. Typically, a blood sample of about 5 milliliters is taken from a patient after a ten to twelve hour fast. The patient repeats the fast another day and provides another blood sample. A patient without diabetes would typically produce a result of between about 80 mg/dl and about 120 mg/dl (or about 4 mmol/L to about 7 mmol/L). If a patient has two fasting glucose levels of 126 mg/dl (or 7 mmol/L), then the patient is typically diagnosed with diabetes.

Similar to the fasting glucose test is the oral glucose tolerance test (OGTT). The patient fasts for ten to twelve hours, then a blood sample is taken. A glucose dose of about 75 milligrams (or 100 milligrams if testing for gestational diabetes) is administered to the patient and blood samples are taken every thirty minutes for the next two hours. If the patient has a glucose level that is equal to or greater than 200 mg/dl (or 11.1 mmol/L), then the patient is diagnosed with diabetes.

An alternative to the fasting method is the two-hour postprandial plasma glucose (2 hrPPG) test. This test measures the amount of glucose in blood plasma after a person eats a meal loaded with a specific amount of sugar, typically about 75 milligrams. After two hours, the patient's glucose levels are ascertained by evaluating a blood sample. If a patient has a level of about 200 mg/dl (or 11.1 mmol/L), then the patient is diabetic.

An alternative to measuring the glucose levels in the blood is measuring certain hemoglobin levels. While this test is not traditionally used for diagnosis, it is indicative of a patient's susceptibility to diabetes. Specifically, the level of hemoglobin A1c (or HbA1c) is measured. About 90% of hemoglobin is hemoglobin A and about 8% of hemoglobin A is made up of minor components referred to as A1c, A1b, A1a1, and A1a2.

Glucose binds to HbA1c and therefore HbA1c levels depend on the blood glucose concentration. That is, the higher the glucose concentration in blood, the higher the level of HbA1c. This test has an advantage over the other tests in that the levels of HbA1c are not affected by short-term fluctuations in blood glucose. HbA1c can be easily measured by high performance liquid chromatography (HPLC) because it carries a different charge and also differs in size compared to other hemoglobin. Non-diabetic patients exhibit HbA1c levels of less than 7% of total hemoglobin. Levels above 7% indicate diabetes.

Even if no specific diagnostic test is administered, a patient is diabetic if the patient exhibits a random blood glucose of greater than 200 mg/dl (or 11.1 mmol/L) and exhibits known symptoms of diabetes, as described above.

As referred to herein, the term “dual inhibitor” refers to a compound that inhibits any two lipoxygenase enzymes, particularly 5- and 12/15-lipoxygenase enzymes or 5- and 15-lipoxygenase enzymes. It is contemplated that one or more dual inhibitors are administered to a subject. To determine if a compound inhibits lipoxygenase enzymes, the compound may be assayed, for example, as detailed in Examples 4 and 5. Other lipoxygenase enzyme assays known in the art may also be employed. A compound is considered to be a dual inhibitor if the compound exhibits an IC₅₀ value of less than 5 micromolar in both a 5-lipoxygenase (5-LO) enzyme assay and in a 12/15-lipoxygenase (12/15-LO) enzyme assay or in a 5-lipoxygenase (5-LO) enzyme assay and in a 15-lipoxygenase (15-LO) enzyme assay. The term “IC₅₀” refers to the concentration of an inhibitor that is required for 50% inhibition of an enzyme in vitro.

It is contemplated that dual inhibitors of the invention, include, but are not limited to compounds of Formula I and/or Formula II.

It has been demonstrated that dual inhibitors of this invention include, but are not limited to Compounds A, D, and E. The structures for Compounds A-E are provided in the table below.

| Compound | Structure | Name | Source |
|---|---|---|---|
| A | 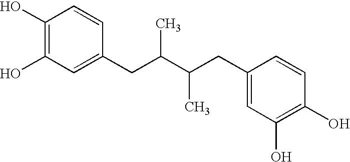 | Nordihydroguaiaretic acid (NDGA) | Sigma-Aldrich |
| B | 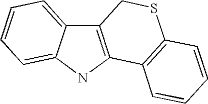 | PD 146176 | Sigma-Aldrich |
| C | 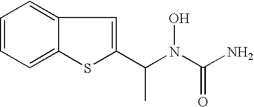 | Zileuton | Chem Pacific USA |
| D | 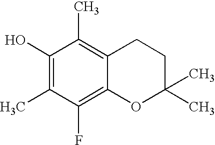 | 8-fluoro-2,2,5,7- tetramethylchroman- 6-ol | Synthesized according to Example 1 |
| E | 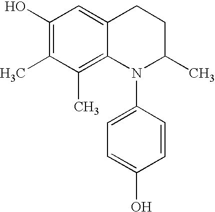 | 1-(4-hydroxyphenyl)- 2,7,8-trimethyl-1,2,3,4- tetrahydroquinolin-6-ol | Synthesized according to Example 2 |

In one embodiment of the invention, Compound A, D, and/or E is administered with one or more compounds of Formula I or II.

“Pharmaceutically effective amount” or “therapeutically effective amount” refers to that amount of a compound of the present invention that is sufficient to effect treatment, as defined below, when administered to a mammal in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can readily be determined by one of ordinary skill in the art.

“Treatment” or “treating” means any treatment of a disease or disorder in a mammal, including:
preventing or protecting against the disease or disorder, that is, causing the clinical symptoms not to develop;
inhibiting the disease or disorder, that is, arresting or suppressing the development of clinical symptoms; and/or
relieving the disease or disorder that is causing the regression of clinical symptoms.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein, the term “prophylaxis” is intended as an element of “treatment” to encompass both “preventing” and “suppressing” as defined herein.

“Protection,” as used herein, is meant to include “prophylaxis.”

It is also contemplated that the composition comprising a pharmaceutically effective amount of one or more dual inhibitors that inhibit both 5-LO and 12/15-LO or both 5-LO and 15-LO may be coadministered with another diabetic medication. What is meant by “coadministered” is the two agents are in clinical association with one another. Coadministration can include administering the agents together or sequentially. The coadministration may take place by the same delivery route or may be by separate delivery routes, for example, the composition comprising the dual inhibitor may be administered peritoneally while the diabetic medication may be administered orally.

What is meant by “diabetic medication” is a medication or pharmaceutical that prevents or treats diabetes or ameliorates the symptoms of diabetes. Diabetic medications, include, but are not limited to pioglitizone, glimepiride, rosiglitazone, glipizide, metforministol, miglitol, repaglinide, acarbose, troglitazone, nateglinide, and combinations thereof.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl,” as defined below.

It will be understood by those skilled in the art with respect to any chemical group containing one or more substituents that such groups are not intended to introduce any substitution or substitution patterns that are sterically impractical and/or physically non-feasible.

The term “acyl” refers to the group —C(O)—R¹⁰, where R¹⁰ may be selected from alkyl, substituted alkyl, alkenyl, substituted alkenyl, alkynyl, substituted alkynyl, cycloalkyl, substituted cycloalkyl, aryl, substituted aryl, heterocyclic, and substituted heterocyclic.

The term “acyloxy” refers to the group —O-acyl, where acyl is defined herein.

The term “alkenyl” refers to a monoradical branched or unbranched, unsaturated or polyunsaturated hydrocarbon chain, having from about 2 to 20 carbon atoms, for example about 2 to 10 carbon atoms and having at least 1 site, and preferably 1-5 sites, of alkenyl unsaturation. This term is exemplified by groups such as ethenyl, but-2-enyl, 3-methyl-but-2-enyl (also referred to as “prenyl”), octa-2,6-dienyl, 3,7-dimethyl-octa-2,6-dienyl (also referred to as “geranyl”), 4,8,12-trimethyl-trideca-3,7,11-trienyl and the like.

The term “alkenylene” refers to a diradical derived from the above defined monoradical, alkenyl.

The term “substituted alkenyl” refers to an alkenyl group in which 1 or more (up to about 5, for example about 3) hydrogen atoms is replaced by a substituent independently selected from the group: oxo (═O), ═S, acyl, acyloxy, optionally substituted alkoxy, optionally substituted amino (wherein the amino group may be a cyclic amine), azido, carboxyl, (optionally substituted alkoxy)carbonyl, (optionally substituted amino)carbonyl, cyano, optionally substituted cycloalkyl, optionally substituted cycloalkenyl, optionally substituted heterocyclic, halogen, hydroxyl (—OH), nitro (—NO₂), sulfamoyl (—SO₂NH₂), sulfanyl or thiol (—SH), sulfinyl (—S(O)H, sulfonyl (—SO₂H), and sulfonic acid (—SO₂OH).

The term “alkoxy” refers to the groups —O-alkyl, —O-alkenyl, —O-cycloalkyl, —O-cycloalkenyl, and —O-alkynyl. Exemplary alkoxy groups are —O-alkyl and include, by way of example, methoxy, ethoxy, n-propoxy, iso-propoxy, n-butoxy, tert-butoxy, sec-butoxy, n-pentoxy, n-hexoxy, 1,2-dimethylbutoxy, and the like.

The term “substituted alkoxy” refers to an alkoxy group wherein at least 1, and preferably 1 to 5, hydrogen atoms are replaced by a substituted selected from those listed with substituted alkyl.

The term “alkyl” refers to a monoradical branched or unbranched saturated hydrocarbon chain having from about 1 to 20 carbon atoms, for example about 1 to 10 carbon atoms. The term “alkyl” also means a combination of linear or branched and cyclic saturated hydrocarbon radical consisting solely of carbon and hydrogen atoms. This term is exemplified by groups such as methyl, ethyl, n-propyl, iso-propyl, n-butyl, iso-butyl, n-hexyl, n-decyl, tetradecyl, 4,8,12-trimethyltridecyl, and the like.

The term “alkylene” refers to a diradical derived from the above-defined monoradical, alkyl.

The term “alkylsulfonyl” refers to —SO₂-alkyl.

The term “substituted alkyl” refers to an alkyl group in which 1 or more (up to about 5, for example about 3) hydrogen atoms is replaced by a substituent independently selected from the group: ═O, ═S, acyl, acyloxy, optionally substituted alkoxy, optionally substituted amino (wherein the amino group may be a cyclic amine), azido, carboxyl, (optionally substituted alkoxy)carbonyl, amido, cyano, optionally substituted cycloalkyl, optionally substituted cycloalkenyl, halogen, hydroxyl, nitro, sulfamoyl, sulfanyl, sulfinyl, sulfonyl, and sulfonic acid. Some of the optional substituents for alkyl are hydroxy, halogen exemplified by chloro and bromo, acyl exemplified by methylcarbonyl; alkoxy, and heterocyclyl exemplified by morpholino and piperidino.

The term “substituted alkylene” refers to a diradical derived from the above-defined monoradical, substituted alkyl.

The term “alkynyl” refers to a monoradical branched or unbranched, unsaturated or polyunsaturated hydrocarbon chain, having from about 2 to 20 carbon atoms, for example about 2 to 10 carbon atoms and having at least 1 site of alkynyl unsaturation and preferably 1 to 5 sites of alkynyl unsaturation.

The term “substituted alkynyl” refers to an alkynyl group having one or more hydrogen groups replaced by a substituent selected from those described above in the definition for substituted alkyl.

The term “substituted alkenylene” refers to a diradical derived from the above-defined monoradical, substituted alkenyl.

The term “amido” refers to the moiety aminoacyl and acylamino.

The term “amino” refers to —NH₂.

The term “substituted amino” refers to —NR¹¹R¹², wherein and R¹¹ and R¹² are independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic, cyano, —SO₂-alkyl, —SO₂-substituted alkyl, and where R¹¹ and R¹² are joined, together with the nitrogen atom bound thereto to form a heterocyclic or substituted heterocyclic group provided that at least one of R¹¹ and R¹² is not hydrogen.

The term “acylamino” refers to the group —NR¹¹—C(O)—R¹⁰. The term “aminoacyl” refers to the group —C(O)—NR¹¹R¹². R¹⁰, R¹¹, and R¹² are as defined herein,

The term “aminosulfonyl” refers to the group —SO₂NR¹¹R¹², wherein R¹¹ and R¹² are independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic, cyano, and where R¹¹ and R¹² are joined, together with the nitrogen atom bound thereto to form a heterocyclic or substituted heterocyclic group provided that at least one of R¹¹ and R¹² is not hydrogen.

The term “sulfonylamino” refers to the group —NR¹¹SO₂R¹², wherein R¹¹ and R¹² are independently selected from the group consisting of hydrogen, alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic, cyano.

The term “aryl” refers to an aromatic cyclic hydrocarbon group of from 6 to 20 carbon atoms having a single ring (e.g., phenyl) or multiple condensed (fused) rings (e.g., naphthyl or anthryl). Exemplary aryls include phenyl, naphthyl and the like.

The term “substituted aryl” refers to an aryl group as defined above, which unless otherwise constrained by the definition for the aryl substituent, is substituted with from 1 to 5 substituents, for example 1 to 3 substituents, independently selected from the group consisting of: hydroxy, thiol, acyl, acyloxy, optionally substituted alkenyl, optionally substituted alkoxy, optionally substituted alkyl (such as tri-halomethyl), optionally substituted alkynyl, optionally substituted amino, optionally substituted aryl, optionally substituted aryloxy, azido, carboxyl, (optionally substituted alkoxy)carbonyl, amido, cyano, optionally substituted cycloalkyl, optionally substituted cycloalkenyl, halogen, optionally substituted heterocyclyl, optionally substituted heterocyclooxy, hydroxyl, nitro, sulfanyl, sulfinyl, and sulfonic acid. Exemplary aryl substituents include alkyl, alkenyl, hydroxy, alkoxy, carboxy, amido, sulfonyl, aminosulfonyl, oxo, cyano, nitro or halogen.

The term “aryloxy” refers to the moiety —O-aryl, where aryl is defined above.

The term “substituted aryloxy” refers to the group —O-substituted aryl.

The term “arylsulfonyl” refers to the moiety —SO₂-aryl, where aryl is defined above.

The term “carboxy” or “carboxyl” refers to the moiety “—C(O)OH,” which may also illustrated as “—COOH.”

The term “cycloalkyl” refers to a cyclic alkyl groups of from 3 to 10 carbon atoms having single or multiple cyclic rings including, by way of example, adamantyl, cyclopropyl, cyclobutyl, cyclopentyl, cyclooctyl, and the like.

The term “substituted cycloalkyl” refers to cyclic alkyl groups when at least 1, and preferably 1 to 5, hydrogen atoms are replaced by a substituent selected from those listed for substituted alkyl.

The term “cycloalkenyl” refers to cyclic alkenyl groups of from 3 to 10 carbon atoms having single or multiple cyclic rings.

The term “halo” or “halogen” refers to fluoro, chloro, bromo and iodo.

The term “heteroaryl” refers to an aromatic group of from 1 to 10 carbon atoms and 1 to 4 heteroatoms selected from oxygen, nitrogen, and sulfur within the ring. Such heteroaryl groups can have a single ring or multiple condensed rings wherein the condensed rings may or may not be aromatic and/or contain a heteroatom provided that the point of attachment is through an atom of the aromatic heteroaryl group. In one embodiment, the nitrogen and/or the sulfur ring atoms can be optionally oxidized to provide for the N-oxide (N→O), sulfinyl, or sulfonyl moieties.

The term “substituted heteroaryl” refers to a heteroaryl group wherein at least one of the hydrogens are replaced by at least one substituent selected from those listed for substituted aryl.

The terms “heterocycle,” “heterocyclic,” “heterocyclo,” and “heterocyclyl” refer to a monovalent, saturated, partially unsaturated or unsaturated (aromatic), carbocyclic radical having one or more rings incorporating one, two, three or four heteroatoms within the ring (chosen from nitrogen, oxygen, and/or sulfur). Exemplary heterocycles include morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, quinuclidine, and the like.

The terms “substituted heterocycle,” “substituted heterocyclic,” “substituted heterocyclo” and “substituted heterocyclyl” refer to a heterocycle group as defined above, which unless otherwise constrained by the definition for the heterocycle, is substituted with from 1 to 5 substituents, for example 1 to 3 substituents, independently selected from the group consisting of: hydroxy, thiol, acyl, acyloxy, optionally substituted alkenyl, optionally substituted alkoxy, optionally substituted alkyl (such as tri-halomethyl), optionally substituted alkynyl, optionally substituted amino, optionally substituted aryl, optionally substituted aryloxy, azido, carboxyl, (optionally substituted alkoxy)carbonyl, amido, cyano, optionally substituted cycloalkyl, optionally substituted cycloalkenyl, halogen, optionally substituted heterocyclyl, optionally substituted heterocyclooxy, hydroxyl, nitro, sulfanyl, sulfinyl, and sulfonic acid.

The term “heterocyclyloxy” refers to the moiety —O-heterocyclyl. The term “substituted heterocyclyloxy” refers to the moiety —O-substituted heterocyclyl.

The term “heterocyclylsulfonyl” refers to the moiety —SO₂-heterocyclyl.

The term “isomers” or “stereoisomers” relates to compounds that have identical molecular formulae but that differ in the arrangement of their atoms in space. Stereoisomers that are not mirror images of one another are termed “diastereoisomers” and stereoisomers that are non-superimposable mirror images are termed “enantiomers,” or sometimes optical isomers. A carbon atom bonded to four non-identical substituents is termed a “chiral center.” Certain compounds of the present invention have one or more chiral centers and therefore may exist as either individual stereoisomers or as a mixture of stereoisomers. Configurations of stereoisomers that owe their existence to hindered rotation about double bonds are differentiated by their prefixes cis and trans (or Z and E), which indicate that the groups are on the same side (cis or Z) or on opposite sides (trans or E) of the double bond in the molecule according to the Cahn-Ingold-Prelog rules. This invention includes all possible stereoisomers as individual stereoisomers or as a mixture of stereoisomers.

### Synthesis of the Compounds of the Invention

Some compounds of the invention may be prepared as described in co-owned U.S. application Ser. No. 10/202,670 (US Publication No. 2003/0073712) filed Jul. 23, 2002 and in co-owned U.S. application Ser. No. 10/941,125 (US Publication No. US 2005/0065150) filed Sep. 15, 2004, incorporated herein by reference in their entirety. Other compounds may be available commercially or from natural sources.

### Utility, Testing and Administration

General Utility

The methods of the present invention are useful in treating diabetes, particularly in lowering (or controlling) elevated blood glucose in a subject in need of such treatment. The methods of the present invention are also useful in lowering elevated blood glucose in a subject suffering from hyperglycemia.

Testing

This section describes how compositions incorporating compositions of the present invention are selected using in vitro and in vivo animal models, and used as therapeutic interventions in diabetic indications.

In vitro evaluation of the ability of a composition to inhibit the enzymes 5-lipoxygenase, 15-lipoxygenase, or 12/15-lipoxygenase as described in Walidge, N. B. et al. Anal. Biochem., Vol. 231 (1995), pp. 354-358 using a high throughput calorimetric method; as well as in vitro evaluation of inhibiting LTB₄ is described in Examples.

In vivo evaluation of the role of dual inhibitor of both 5-LO and 12/15-LO enzymes in the reduction of serum glucose levels and insulin was performed in C57BLKs/J-m+/+Lepr db mice, a non-insulin dependent diabetic mellitus (NIDDM) model, as described in Examples. Dual inhibitors of both 5 and 12/15-lipoxygenase enzymes are able to improve glucose control in animal models of diabetes and demonstrated a significant lowering of the baseline serum glucose levels compared to selective 5-LO and 12/15-LO inhibitors. All of the inhibitors at their higher dose tested decreased baseline serum glucose levels between 8 to 26% with a p<0.05 compared to the vehicle group. Dual inhibitors such as Compounds A, D, and E and certain compounds of Formula I and Formula II demonstrated an effect of 12-26% serum glucose lowering while selective known 5-lipoxygenase and selective known 12/15-lipoxygenase compounds lowered the baseline glucose by 8 and 10%.

Administration

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for nonliquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition. Easton, Pa., Mack Publishing Company, 1990.

The compounds of the present invention are administered at a therapeutically or pharmaceutically effective dosage, e.g., a dosage sufficient to provide treatment for diabetes as previously described. Administration of the compounds or pharmaceutical compositions thereof for practicing the present invention can be by any method that delivers the compounds systemically and/or locally. These methods include oral routes, parenteral routes, intraduodenal routes, etc.

While human dosage levels have yet to be optimized for the compounds of the invention, generally, a daily dose is from about 0.01 to 10.0 mg/kg of body weight, for example about 0.1 to 5.0 mg/kg of body weight. The precise effective amount will vary from subject to subject and will depend upon the species, age, the subject's size and health, the nature and extent of the condition being treated, recommendations of the treating physician, and the therapeutics or combination of therapeutics selected for administration. The subject may be administered as many doses as is required to reduce and/or alleviate the signs, symptoms, or causes of the disorder in question, or bring about any other desired alteration of a biological system

In employing the compounds of this invention for treatment of diabetes, any pharmaceutically acceptable mode of administration can be used with other pharmaceutically acceptable excipients, including solid, semi-solid, liquid or aerosol dosage forms, such as, for example, tablets, capsules, powders, liquids, suspensions, suppositories, aerosols or the like. The compounds of this invention can also be administered in sustained or controlled release dosage forms, including depot injections, osmotic pumps, pills, transdermal (including electrotransport) patches, and the like, for the prolonged administration of the compound at a predetermined rate, preferably in unit dosage forms suitable for single administration of precise dosages.

The compounds of this invention may also be administered as compositions prepared as foods for foods or animals, including medical foods, functional food, special nutrition foods and dietary supplements. A “medical food” is a product prescribed by a physician that is intended for the specific dietary management of a disorder or health condition for which distinctive nutritional requirements exist, and may include formulations fed through a feeding tube (referred to as enteral administration or gavage administration). A “dietary supplement” shall mean a product that is intended to supplement the human diet and may be provided in the form of a pill, capsule, tablet, or like formulation. By way of example, but not limitation, a dietary supplement may include one or more of the following dietary ingredients: vitamins, minerals, herbs, botanicals, amino acids, and dietary substances intended to supplement the diet by increasing total dietary intake, or a concentrate, metabolite, constituent, extract, or combinations of these ingredients, not intended as a conventional food or as the sole item of a meal or diet. Dietary supplements may also be incorporated into food stuffs, such as functional foods designed to promote control of glucose levels. A “functional food” is an ordinary food that has one or more components or ingredients incorporated into it to give a specific medical of physiological benefit, other than a purely nutritional effect. “Special nutrition food” means ingredients designed for particular diet related to conditions or to support treatment of nutritional deficiencies.

Generally, depending on the intended mode of administration, the pharmaceutically acceptable composition will contain about 0.1% to 90%, for example about 0.5% to 50%, by weight of a compound or salt of compound of the present invention, the remainder being suitable pharmaceutical excipients, carriers, etc.

One manner of administration for the conditions detailed above is oral, using a convenient daily dosage regimen which can be adjusted according to the degree of affliction. For such oral administration, a pharmaceutically acceptable, non-toxic composition is formed by the incorporation of any of the normally employed excipients, such as, for example, mannitol, lactose, starch, magnesium stearate, sodium saccharine, talcum, cellulose, sodium crosscarmellose, glucose, gelatin, sucrose, magnesium carbonate, and the like. Such compositions take the form of solutions, suspensions, tablets, dispersible tablets, pills, capsules, powders, sustained release formulations and the like.

The compositions will take the form of a pill or tablet and thus the composition will contain, along with the active ingredient, a diluent such as lactose, sucrose, dicalcium phosphate, or the like; a lubricant such as magnesium stearate or the like; and a binder such as starch, gum acacia, polyvinylpyrrolidine, gelatin, cellulose and derivatives thereof, and the like.

Liquid pharmaceutically administrable compositions can, for example, be prepared by dissolving, dispersing, etc. an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, sodium acetate, sodium citrate, cyclodextrine derivatives, sorbitan monolaurate, triethanolamine acetate, triethanolamine oleate, etc.

For oral administration, a pharmaceutically acceptable non-toxic composition may be formed by the incorporation of any of the normally employed excipients, such as, for example, pharmaceutical grades of mannitol, lactose, starch, magnesium stearate, talcum, cellulose derivatives, sodium crosscarmellose, glucose, sucrose, magnesium carbonate, sodium saccharin, talcum and the like. Such compositions take the form of solutions, suspensions, tablets, capsules, powders, sustained release formulations and the like.

For a solid dosage form, the solution or suspension in, for example, propylene carbonate, vegetable oils or triglycerides, may be encapsulated in a gelatin capsule. Such diester solutions, and the preparation and encapsulation thereof, are disclosed in U.S. Pat. Nos. 4,328,245; 4,409,239; and 4,410,545. For a liquid dosage form, the solution, e.g. in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active compound or salt in vegetable oils, glycols, triglycerides, propylene glycol esters (e.g. propylene carbonate) and the like, and encapsulating these solutions or suspensions in hard or soft gelatin capsule shells.

Other useful formulations include those set forth in U.S. Pat. Nos. Re. 28,819 and 4,358,603.

Another manner of administration is parenteral administration, generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents, solubility enhancers, and the like, such as for example, sodium acetate, sorbitan monolaurate, triethanolamine oleate, cyclodextrins, etc.

Another approach for parenteral administration employs the implantation of a slow-release or sustained-release system, such that a constant level of dosage is maintained. The percentage of active compound contained in such parenteral compositions is highly dependent on the specific nature thereof, as well as the activity of the compound and the needs of the subject. However, percentages of active ingredient of 0.01% to 10% in solution are employable, and will be higher if the composition is a solid which will be subsequently diluted to the above percentages. The composition may comprise 0.2% to 2% of the active agent in solution.

Nasal solutions of the active compound alone or in combination with other pharmaceutically acceptable excipients can also be administered.

Formulations of the active compound or a salt may also be administered to the respiratory tract as an aerosol or solution for a nebulizer, or as a microfine powder for insufflation, alone or in combination with an inert carrier such as lactose. In such a case, the particles of the formulation have diameters of less than 50 microns, for example less than 10 microns.

### EXAMPLES

The following preparations and examples are given to enable those skilled in the art to more clearly understand and to practice the present invention. They should not be considered as limiting the scope of the invention, but merely as being illustrative and representative thereof.

Abbreviations used in the examples have the following meanings unless stated otherwise:

AcOH=acetic acid

aq.=aqueous

conc.=concentrated

br s=broad singlet

d=doublet

EtOAc=ethyl acetate

g=gram

h=hour

HPLC=high performance liquid chromatography

Hz=Hertz

kg=kilograms

m/z=mass to charge ratio

MeOH=methanol

mg=milligrams

MHz=mega Hertz

min=minute

mL=milliliter

mM=millimolar

mmol=millimole

MS mass spectroscopy

N=normal

nm=nanometer

NMR=nuclear magnetic resonance

ppm=parts per million

rpm=revolutions per minute

s=singlet

sec=second

t=triplet

μL=microliter

v/v=volume/volume

μM=micromolar

### Example 1

### Synthesis of Compound D

### 8-fluoro-2,2,5,7-tetramethylchroman-6-ol

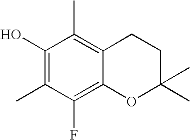

### Step 1: 2,2,5,7-Tetramethyl-8-nitrochroman-6-yl acetate

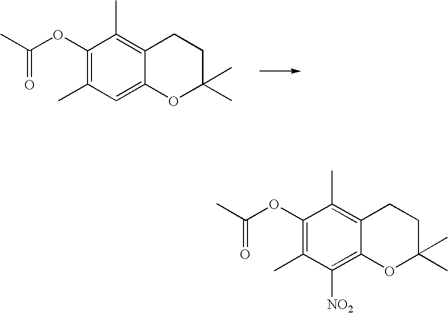

A solution of 2,2,5,7-tetramethylchroman-6-yl acetate (4.23 g) in AcOH (120 mL) was treated with conc. HNO₃ (10.7 mL) and stirred for 1 h. The reaction mixture was then partitioned between EtOAc and H₂O, the organic layer collected, dried with Na₂SO₄ and the solvents evaporated yielding 2,2,5,7-tetramethyl-8-nitrochroman-6-yl acetate as a yellow solid (4.91 g). MS: m/z=294.1 (M+H)⁺.

### Step 2: 8-Amino-2, 2, 5, 7-tetramethylchroman-6-yl acetate

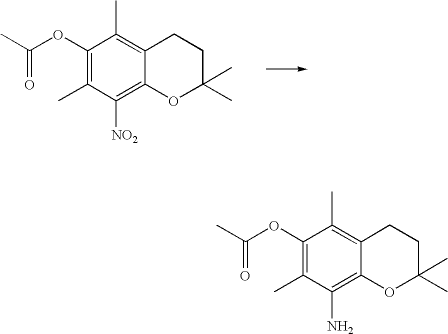

A solution of 2,2,5,7-tetramethyl-8-nitrochroman-6-yl acetate (4.51 g) in MeOH (300 mL) and H₂O (53 mL) was treated with NH₄Cl (12.42 g) and iron powder (7.09 g) and stirred at 50° C. for 14 h followed by 2 h at 80° C. The reaction mixture was allowed to cool to ambient temperature and filtered. Upon solvent evaporation, the extraction was carried out with H₂O and EtOAc. The organic layer was collected, dried with Na₂SO₄ and the solvents evaporated, and the residue was subjected to column chromatography (SiO₂: hexane:EtOAc) yielding 8-amino-2,2,5,7-tetramethyl-chroman-6-yl acetate as a white solid (3.02 g). ¹H-NMR (300 MHz, CDCl₃,) δ=: 2.63 (t, J=7 Hz, 2H), 2.36 (s, 3H), 1.97 (s, 6H), 1.83 (t, J=7 Hz, 2H), 1.35 (s, 6H) ppm. ¹³C NMR (75 MHz, CDCl₃,) δ=169.8, 140.6, 139.5, 132.2, 117.0, 116.7, 112.6, 73.5, 32.9, 20.7, 20.6, 11.7, 10.6 ppm. MS: m/z=264.2 (M+H)⁺

### Step 3: 8-Fluoro-2,2,5,7-tetramethylchroman-6-yl acetate

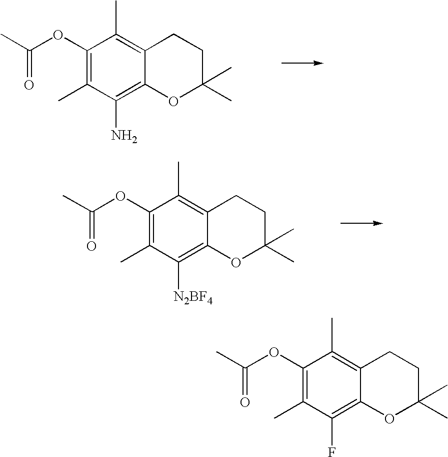

To a solution of 8-amino-2,2,5,7-tetramethyl-chroman-6-yl acetate (2.55 g) in H₂O (32 mL) and conc. HCl (5.34 mL) at 0° C., was added a cooled solution of NaNO₂ (742 mg, 10.8 mmol) in H₂O (5.34 mL) and the reaction mixture was stirred for 1 h at 0° C. Subsequent addition of 40% aq. HBF₄ (10.7 mL) resulted in precipitation of a yellow solid, which was collected by filtration after 10 min stirring. After removing traces of water by lyophilization the yellow solid (3.18 g) was crushed into powder, placed into a flask that was submerged for 20 sec into an oil bath preheated to 200° C. As soon as the reaction started (as indicated by formation of brown oil and fumes) the flask was taken out of the oil bath and the reaction was allowed to self-propagate and eventually cool to ambient temperature. Column chromatography (SiO₂: hexane:CH₂Cl₂, 100%) yielded 8-fluoro-2,2,5,7-tetramethylchroman-6-yl acetate as a yellow oil (946 mg) MS: m/z=267.2 (M+H)⁺

### Step 4: 8-Fluoro-2,2,5,7-tetramethylchroman-6-ol

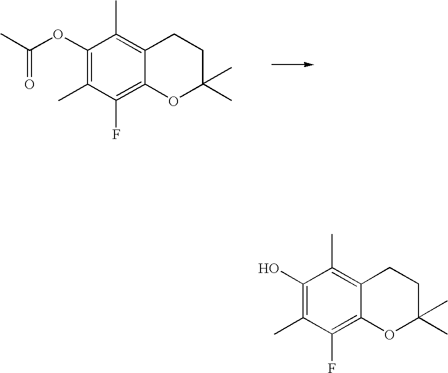

A solution of 8-fluoro-2,2,5,7-tetramethylchroman-6-yl acetate (946 mg) in MeOH (100 mL) was treated with K₂CO₃ (2.46 g) and the resulting suspension was stirred vigorously for 1 h, followed by addition of H₂O and weak HCl to make the solution neutral. Partial solvents evaporation was followed by extraction with EtOAc. Organic layer was collected and dried with Na₂SO₄, followed by solvent evaporation. Column chromatography (SiO₂: hexane:CH₂Cl₂, 1:1 v/v) yielded 8-fluoro-2,2,5,7-tetramethylchroman-6-ol a white solid (376 mg). ¹H-NMR (300 MHz, CDCl₃,) δ=4.40 (s, 1H), 2.65 (t, J=7 Hz, 2H), 2.18 (d, J=2 Hz, 3H), 2.11 (d, J=0.9 Hz, 3H), 1.86 (t, J=7 Hz, 2H), 1.37 (s, 6H) ppm. ¹⁹F NMR (282.4 MHz, CDCl₃) δ=142.7 ppm. MS: m/z=225.1(M+H)⁺, 247.1(M+Na)⁺.

### Example 2

### Synthesis of Compound E

### 1-(4-Hydroxyphenyl)-2,7,8-trimethyl-1,2,3,4-tetrahydroquinolin-6-ol

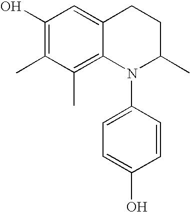

### Step 1: 5-Bromo-2,3-dimethylbenzene-1,4-diol

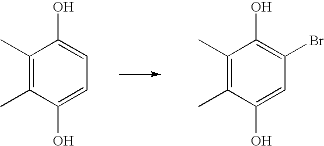

To a solution of 2,3 dimethyl hydroquinone (4 g) in dry ether, was added dry bromine (1.63 mL), dropwise at 0° C. The reaction mixture was stirred at this temperature for 2 hours, followed by quenching with sodium thiosulphate solution. The organic extract was separated out, dried, concentrated and purified over silica (1% ethyl acetate-hexane to obtain 5-bromo-2,3-dimethylbenzene-1,4-diol as white solid (4.7 g). ¹H-NMR (400 MHz, CDCl₃) δ=2.11 (s, 3H), 2.22 (s, 3H), 4.43 (s, 1H), 5.15 (s, 1H), 6.78 (s, 1H) ppm.

### Step 2: 5-Bromo-2,3-dimethyl-1,4-bis(benzyloxy)-benzene

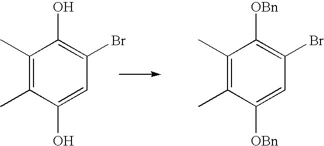

A solution of 5-bromo-2,3-dimethylbenzene-1,4-diol (4.5 g) in dry acetone (150 mL) was refluxed with benzyl bromide (9.9 mL) in presence of anhydrous potassium carbonate (11.5 g) and catalytic sodium iodide under argon for 10 hours. The mixture was filtered off, acetone removed and the material was purified over silica (1% ethyl acetate-hexane) to obtain 5-bromo-2,3-dimethyl-1,4-bis(benzyloxy)-benzene (4.5 g) as a white solid. ¹H-NMR (400 MHz, CDCl₃) δ=2.16 (s, 3H), 2.25 (s, 3H), 4.85 (s, 2H), 5.01 (s, 2H), 7.00 (s, 1H), 7.38-7.53 (m, 8H), 7.55 (d, 2H) ppm.

### Step 3: 4-(2,5-Bis(benzyloxy)-3,4-dimethylphenyl)butan-2-one

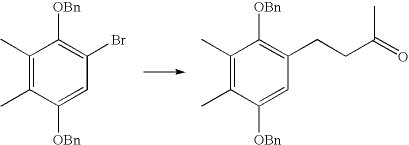

A solution of 5-bromo-2,3-dimethyl-1,4-bis(benzyloxy)-benzene (5 g) in dry acetonitrile (150 mL) was treated with methyl vinyl ketone (1.98 mL), pyridine (1.42 mL), zinc dust (1.9 g) and the mixture thoroughly deaerated. Thereafter nickel chloride hexahydrate (598 mg) was added. The mixture was refluxed for 8 hours under argon, cooled, filtered, concentrated and purified over silica (10% ethyl acetate-hexane to obtain 4-(2,5-bis(benzyloxy)-3,4-dimethylphenyl)butan-2-one (1.2 g) as a white solid. ¹H-NMR (400 MHz, CDCl₃) δ=2.06 (s, 3H), 2.19 (s, 3H), 2.25 (s, 3H), 2.71 (t, 2H, J=7.2 Hz), 2.86 (t, 2H, J=7.2 Hz), 4.73 (s, 2H), 5.01 (s, 2H), 6.61 (s, 1H), 7.31-7.47 (m, 10H) ppm.

### Step 4: N-(4-(2,5-bis(benzyloxy)-3,4-dimethylphenyl)butan-2-yl)-4-methoxyaniline

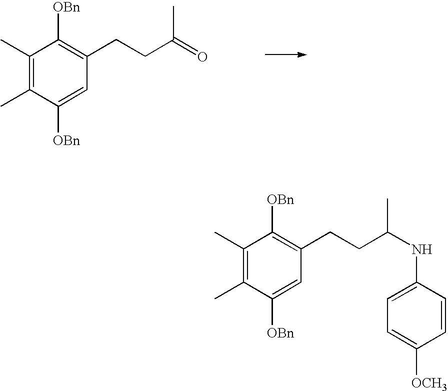

A solution of 4-(2,5-bis(benzyloxy)-3,4-dimethylphenyl)butan-2-one (2 g) in dry dichloroethane (75 mL) was treated with p-anisidine (0.67 g) and sodium cyano borohydride (2.2 g) in presence of glacial acetic acid (0.3 mL). The mixture was stirred for 24 hours under argon and finally quenched with 1N sodium hydroxide (10 mL). The organic layer was separated out, dried, concentrated and finally purified over silica to obtain N-(4-(2,5-bis(benzyloxy)-3,4-dimethylphenyl)butan-2-yl)-4-methoxyaniline (1.5 g) as a viscous liquid. ¹H-NMR (400 MHz, CDCl₃) δ=1.12 (d, 3H), 1.74-1.79 (m, 2H), 2.06 (s, 3H), 2.19 (s, 3H), 2.63-2.65 (m, 1H), 2.76-2.79 (m, 1H), 3.35-3.36 (m, 1H), 3.69 (s, 3H), 4.73 (s, 2H), 4.93 (s, 2H), 6.44 (d, 2H, J=7 Hz), 6.59 (s, 1H), 6.71 (d, 2H, J=6.8 Hz), 7.32-7.47 (m, 10H) ppm. MS: (m/z)=496.5.

### Step 5: 1-(4-Methoxyphenyl)-2,7,8-trimethyl-1,2,3,4-tetrahydroquinolin-6-ol

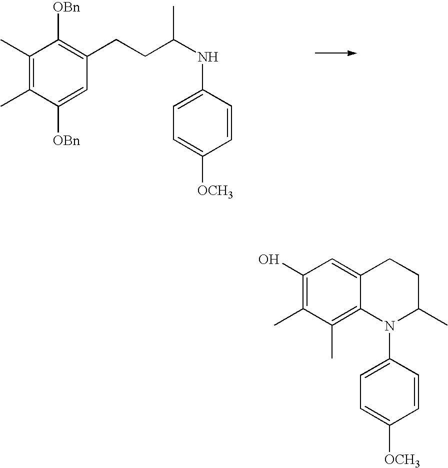

A solution of N-(4-(2,5-bis(benzyloxy)-3,4-dimethylphenyl)butan-2-yl)-4-methoxyaniline (1.7 g) in dry THF (50 mL) was hydrogenolysed in presence of palladium charcoal for 5 hours. The reaction mixture was filtered over Celite® mixed with sodium dithionite under stringent argon atmosphere. The crude filtrate was immediately treated with 2,3-dichloro-5,6-dicyano-1,4-benzoquinone (DDQ) and stirred under argon for 24 hours. Thereafter the solvent was removed and the resultant deep purple viscous mass was purified over silica to obtain a dark purple viscous residue which was subjected to preparative HPLC (Phenomenex LUNA) to obtain 1-(4-methoxyphenyl)-2,7,8-trimethyl-1,2,3,4-tetrahydroquinolin-6-ol (270 mg) as a sticky purple solid. ¹H-NMR (400 MHz, CDCl₃) δ=1.20 (d, 3H), 1.37-1.41 (m, 1H), 1.80 (s, 3H), 2.04-2.07 (m, 1H), 2.10 (s, 3H), 2.58-2.63 (m, 2H), 3.74 (s, 3H), 3.91-3.95 (m, 1H), 4.41 (s, 1H), 6.47 (s, 1H), 6.62 (d, 2H), 6.72 (d, 2H) ppm. MS: (m/z)=298.4.

### Step 6: 1-(4-Hydroxyphenyl)-2,7,8-trimethyl-1,2,3,4-tetrahydroquinolin-6-ol

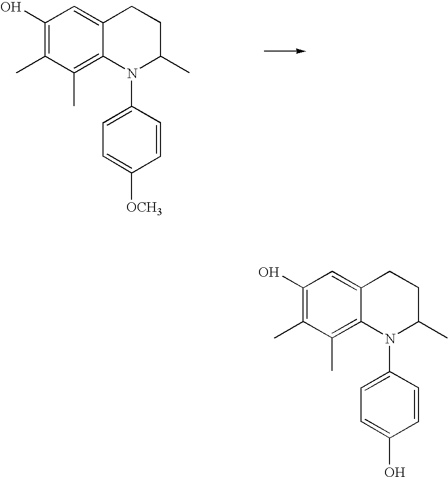

A solution of 1-(4-methoxyphenyl)-2,7,8-trimethyl-1,2,3,4-tetrahydroquinolin-6-ol (270 mg) in dry methylene chloride (5 mL) was treated with boron tribromide (0.25 mL) under argon at −78° C. The reaction mixture was thereafter stirred at room temperature for 3 hours followed by quenching with saturated sodium bicarbonate solution under ice cold condition. The organic layer was separated out, dried and concentrated. The sticky residue was repeatedly washed with dry hexane to obtain 1-(4-hydroxyphenyl)-2,7,8-trimethyl-1,2,3,4-tetrahydroquinolin-6-ol (146 mg) as a light purple solid. ¹H-NMR (d₆-DMSO) δ=1.09 (d, 3H), 1.34 (br s, 1H), 1.68 (s, 3H), 1.86 (br s, 1H), 1.95 (s, 3H), 2.49 (s, 2H), 3.81 (br s, 1H), 6.46 (brs, 3H), 8.80 (brs, 2H) ppm. ¹³C-NMR (100 MHz, d₆-DMSO) δ=12.0, 14.5, 19.4, 23.4, 25.2, 55.9, 111.8, 115.4, 121.1, 121.5, 128.4, 132.4, 144.1, 150.7, 150.9 ppm. MS: (m/z)=284.

### Example 3

### LTB₄-Assay

This procedure was used for measuring the release of the leukotriene LTB4 from a neutrophil cell line using a competitive ELISA technique.

Materials and Equipments

Materials for Cell Preparation and Experiment

MPRO cell line (ATCC, Catalog # CRL-11422)

Calcium ionophore (A23187) (Sigma, Catalog # C7522)

Nordihydroguaiaretic acid (NDGA) (BioMol, Catalog # EI101-0001)

Retinoic Acid (all-trans) (ATRA) (Sigma, Catalog # 95152)

Sterile, tissue-culture treated 96-well plates (Corning, Catalog # 3614)

Materials for LTB4 ELISA

Precoated (Mouse Anti-Rabbit IgG) EIA 96 Well Strip Plates (Cayman, Catalog # 400004)

Leukotriene B4 AChE Tracer (Cayman Catalog # 420110)

Leukotriene B4 EIA Antiserum (Cayman Catalog # 420112)

Ellman's Reagent (Cayman Catalog # 400050)

EIA Buffer Concentrate (10×) (Cayman Catalog # 400060)

Wash Buffer Concentrate (400×) (Cayman Catalog # 400062)

Plastic plate covers (Cayman Catalog # 400012)

Procedure

A mouse promyelocytic cell line (MPRO) was used in this assay. These cells were committed immature neutrophils that can be differentiated into mature neutrophils by treatment with 10 μM all-trans retinoic acid for 72 hours.

Following 72 hours of differentiation, cells were stimulated with 1 μM of a calcium ionophore (A23187) in the presence or absence of test compound or vehicle for 1 hour at 37° C. After this time, supernatant was removed from the cells and the LTB4 levels were determined following manufacturer's instructions, using a Leukotriene B4 EIA kit from Cayman (Cat # 520111).

The negative controls were media samples from differentiated but unstimulated cells. The compounds were screened at 5 concentrations in quadruplicate starting at 10 μM.

Compounds of the present invention were considered to be active if they exhibited inhibition of LTB4 production with an EC₅₀ in a range of less than 1 μM.

### Example 4

### 5-Lipoxygenase Enzyme Assay

This procedure was used for measuring the enzymatic activity of human recombinant 5-lipoxygenase using a colorimetric method based on the ferric oxidation of xylenol orange.

Materials

96 well flat bottom microfilter plates (VWR, Catalog # 62402-933 9295)

Lipoxygenase screening assay buffer (Cayman, Catalog # 760710)

Human recombinant 5-lipoxygenase (Cayman, Catalog # 60402)

Arachidonic Acid (Sigma, Catalog # A3555)

Xylenol orange tetrasodium salt (Aldrich, Catalog # 227854)

Iron (II) sulfate heptahydrate (Sigma, Catalog # F7002)

Sulfuric acid (95-98%) [18M]

Methanol

Procedure

Human recombinant 5-lipoxygenase (Cayman Cat # 60402) was used in this assay. The test compound and/or vehicle was added to 0.5 μL 5-lipoxygenase in 50 mM Tris-HCl buffer, pH 7.4. The reaction was initiated by addition of 70 μM arachidonic acid in Tris-HCl buffer, pH 7.4, and terminated after a 10 minute incubation at room temperature by addition of FOX reagent (25 mM sulphuric acid, 100 μM xylenol orange, 100 μM iron (II) sulphate, methanol:water 9:1). The yellow color of acidified xylenol orange was converted to a blue color by the lipid hydroperoxide-mediated oxidation of Fe²⁺ ions and the interaction of the resulting Fe³⁺ ions with the dye. The complex was allowed to form during a 1 hour incubation at room temperature with shaking. Absorbance of the Fe³⁺ complex was then measured at 620 nm using a spectrophotometer. Negative controls contained enzyme during the incubation step but substrate was not added until after the FOX reagent.

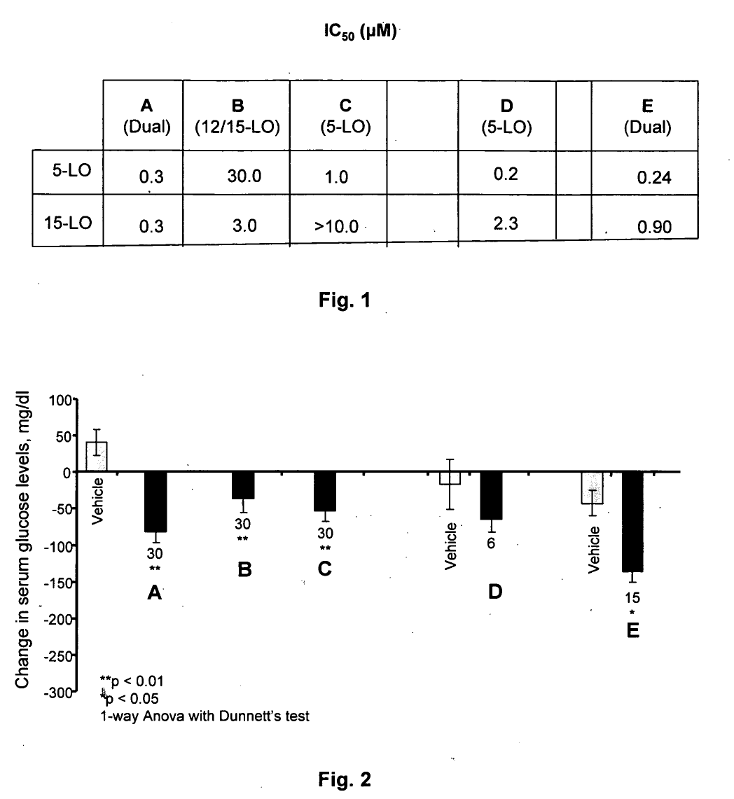

Compounds screened at 5 concentrations in triplicate starting at 10 μM, were considered to be active when they exhibited inhibition of 5-Lipoxygenase with an IC₅₀ of about 5 μM or less. The results for compounds A, B, C, D, and E are presented in FIG. 1.

### Example 5

### 12/15-Lipoxygenase Enzyme Assay

This procedure was used for measuring the enzymatic activity of porcine leukocyte 12/15-lipoxygenase using a colorimetric method based on the ferric oxidation of xylenol orange.

Materials

96 well flat bottom microfilter plates (VWR, Catalog # 62402-933 9295)

Lipoxygenase screening assay buffer (Cayman, Catalog # 760710)

Porcine leukocyte 12/15-lipoxygenase (Cayman, Catalog # 60300)

Arachidonic Acid (Sigma, Catalog # A3555)

Xylenol orange tetrasodium salt (Aldrich, Catalog # 227854)

Iron (II) sulfate heptahydrate (Sigma, Catalog # F7002)

Sulfuric acid (95-98%) [18M]

Methanol

Procedure

Porcine Leukocyte 12/15-lipoxygenase (Cayman Cat # 60300) was used in this assay. The test compound and/or vehicle were added to 1.3 μL 12/15-lipoxygenase in 50 mM Tris-HCl buffer, pH 7.4. The reaction was initiated by addition of 70 μM arachidonic acid in Tris-HCl buffer, pH 7.4, and terminated after a 10 minute incubation at room temperature by addition of FOX reagent (25 mM sulphuric acid, 100 μM xylenol orange, 100 μM iron (II) sulphate, methanol:water 9:1). The yellow color of acidified xylenol orange was converted to a blue color by the lipid hydroperoxide-mediated oxidation of Fe²⁺ ions and the interaction of the resulting Fe³⁺ ions with the dye. The complex was allowed to form during a 1 hour incubation at room temperature with shaking. Absorbance of the Fe³⁺ complex was then measured at 620 nm using a spectrophotometer.

Negative controls contained enzyme during the incubation step but substrate was not added until after the FOX reagent.

Compounds screened at 5 concentrations in triplicate starting at 10 μM were considered to be active when they exhibited inhibition of 12/15-Lipoxygenase with an IC₅₀ of about 5 μM or less. The results for compounds A, B, C, D, and E are presented in FIG. 1.

### Example 6

### Therapeutic Study: Serum Glucose Levels in Diabetic Animals

1. Animals

Non-insulin dependent diabetic mellitus (NIDDM) male mice (C57BLKS/J-m+/+Lepr db), weighing 45±5 g (10 weeks of age) and provided by Institute for Animal Reproduction (IAR, Japan), were used. These animals exhibited hyperinsulinemia, hyperglycemia and islet atrophy.

2. Chemicals

Dimethylsulfate oxide (DMSO, MERCK Germany),

ELISA Insulin assay kit (SPI bio, France),

Glucose (Merck, Germany),

Glucose-HA Assay kit (Wako, Japan),

Methylcellulose (Sigma, USA),

Sodium Chloride (Wako, Japan),

Tween 80 (Wako, Japan)

3. Methods

Groups of 10 non-insulin dependent diabetic mellitus (NIDDM) male mice (C57BLKS/J-m+/+Lepr db), weighing 45±5 g and 10-weeks-old, were used. The animals were housed in Individually Ventilated Cages Racks (IVC Racks) throughout the experiment; all animals were allowed free access to sterilized Lab chow and sterilized distilled water. The test compounds, as well as vehicle (0.5% DMSO/2% Tween 80/0.9% NaCl), were each administered intraperitoneally twice daily (1st at 9:00 A.M. and 2nd at 16:00 P.M.) for a total of 14 consecutive days. Using blood samples collected from the orbital sinus, pre-treatment serum glucose and insulin levels were determined 24 hours prior to the first dosing on day 0. Post-treatment glucose and insulin levels were detected 4 hours after dosing on days 11 and 14. Also, serum glucose and insulin levels were obtained before and 15, 30 and 90 minutes after glucose loading (orally glucose tolerance test (OGTT) 2 g/kg), 4 hours following the dosing on day 11. Serum glucose and Insulin (on days 0, 11 and 14) levels were determined by enzymatic (Mutaratase-GOD) and ELISA (mouse insulin assay kit) methods, respectively. The percentage of post-treatment relative to pre-treatment group values obtained on days 11 and 14 were analyzed using One Way ANOVA followed by Dunnett's test for comparison between vehicle and treated groups. The differences are considered significant at p<0.05.

For effect on OGTT, AUC (area under the curve) for the serum glucose at times 0, 15, 30, and 90 minutes after OGTT was plotted for each test compound and its respective vehicle control. This information is presented in FIG. 2. Statistical analysis was performed using two way ANOVA followed by Bonferroni's test for comparison between vehicle and each treated group. Body weight and food intake were measured daily through the course of the study.

Serum specimens on day 0 and day 14 were obtained by centrifuging blood samples at 3000 rpm 4° C. for 10 minutes. Subsequently, the epididymal fat depots and liver were removed with a surgical excision and was then frozen at −80° C.

Results: The dual inhibitors (compounds A, D, and E) at their higher dose tested decreased baseline serum glucose levels between 12 to 26% with a p<0.05 compared to the vehicle group.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

We claim:

1. A method of preventing or treating diabetes in a subject comprising:
a. identifying a subject susceptible to diabetes; and
b. administering to the subject a composition comprising a pharmaceutically effective amount of one or more dual inhibitors that inhibit both 5-lipoxygenase (5-LO) and 12/15-lipoxygenase (12/15-LO) enzymes or both 5-lipoxygenase (5-LO) and 15-lipoxygenase (15-LO) enzymes.

2. The method of claim 1, wherein the subject is a mammal.

3. The method of claim 2, wherein the subject is a human.

4. The method of claim 1, wherein the subject is susceptible to Type 1 diabetes.

5. The method of claim 1, wherein the subject is susceptible to Type 2 diabetes.

6. The method of claim 1, wherein the dual inhibitor exhibits an in vitro IC₅₀ value of less than 5 micromolar in both a 5-lipoxygenase (5-LO) enzyme assay and a 12/15-lipoxygenase (12/15-LO) enzyme assay or in both a 5-lipoxygenase (5-LO) enzyme assay and a 15-lipoxygenase (15-LO) enzyme assay.

7. The method of claim 1, wherein the dual inhibitor is selected from the group consisting of nordihydroguaiaretic acid (NDGA), 8-fluoro-2,2,5,7-tetramethylchroman-6-ol, and 1-(4-hydroxyphenyl)-2,7,8-trimethyl-1,2,3,4-tetrahydroquinolin-6-ol.

8. The method of claim 7, wherein the dual inhibitor is 1-(4-hydroxyphenyl)-2,7,8-trimethyl-1,2,3,4-tetrahydroquinolin-6-ol.

9. The method of claim 1, wherein the dual inhibitor is a compound of Formula I: 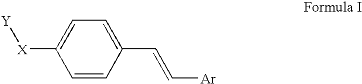
wherein,
Ar is an aryl group optionally substituted with one or more groups independently selected from alkyl, alkenyl, hydroxy, alkoxy, carboxy, amido, sulfonyl, aminosulfonyl, cyano, nitro and halogen;
X is a bond, an alkylene or an alkenylene group, and
Y is nitro, cyano, carboxy, amino, sulfonylamino, aminosulfonyl, alkylsulfonyl, arylsulfonyl, heterocyclylsulfonyl, or heterocyclic selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
or single stereoisomers and mixtures of stereoisomers, and pharmaceutically acceptable salts thereof.

10. The method of claim 9, wherein the compound is selected from the group consisting of:
4-[2-(4-bromo-phenyl)-vinyl]-benzene-1,2-diol;
4-[2-(4-tert-Butyl-phenyl)-vinyl]-1,2-bis-methoxymethoxy-benzene;
1,2-bis-methoxymethoxy-4-[2-(4-trifluoromethyl-phenyl)-vinyl]-benzene;
2,6-dimethyl-4-[2-(4-nitro-phenyl)-vinyl]-phenol;
4-{2-[3,4-dihydroxy-2-(3-methyl-but-2-enyl)-phenyl]-vinyl}benzoic acid methyl ester;
N-{4-[2-(3,4-dihydroxy-phenyl)-vinyl]-phenyl}acetamide;
N-{4-[2-(3,4-dihydroxy-phenyl)-vinyl]-phenyl}methanesulfonamide;
4-{2-[4-(piperidine-1-sulfonyl)-phenyl]-vinyl}benzene-1,2-diol;
4-[2-(2,3,4-trihydroxy-phenyl)-vinyl]-benzonitrile;
5-(4-nitrophenethyl)benzene-1,2,3-triol;
4-(3,4,5-trihydroxyphenethyl)benzonitrile;
5-(4-(methylamino)styryl)-3-(3-methylbut-2-enyl)benzene-1,2-diol;
5-(4-(dimethylamino)styryl)-3-(3-methylbut-2-enyl)benzene-1,2-diol;
4-(2,4-dihydroxystyryl)benzene-1,2-diol;
4-{2-[4-amino-sulfonyl)-phenyl]-vinyl}benzene-1,2-diol;
4-[2-(4-nitro-phenyl)-vinyl]-benzene-1,2-diol; and
4-[2-(3,4-dihydroxy-phenyl)-vinyl]-benzoic acid methyl ester.

11. The method of claim 1, wherein the dual inhibitor is a compound of Formula II: 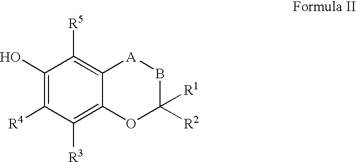
wherein,
R¹ is alkyl optionally substituted with halogen, hydroxy, cyano, amido or carboxy; or alkenyl optionally substituted with halogen, hydroxy, cyano, amido or carboxy;
R² is
alkyl optionally substituted with halogen, hydroxy, cyano, or carboxy;
alkenyl optionally substituted with halogen, hydroxy, cyano, or carboxy;
aryl optionally substituted with one or more groups independently selected from alkyl, alkenyl, hydroxy, alkoxy, carboxy, amido, sulfonyl, aminosulfonyl, cyano, nitro and halogen; or
heterocyclyl selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
R³ is hydrogen, optionally substituted alkyl, halogen, optionally substituted aryl or optionally substituted heterocyclyl;
R⁴ and R⁵ are independently of each other hydrogen, optionally substituted alkyl or halogen, or R⁴ and R⁵ may form an optionally substituted unsaturated or aromatic 5-6 membered ring optionally containing one or more heteroatoms or a C₇₋₁₂ bicyclo [a.b.c]alkyl ring where a, b, and c are 0 to 6, and
-A-B— is —CH₂—(CH₂)₀₋₂— or —CH═CH—;
or single stereoisomers and mixtures of stereoisomers, and pharmaceutically acceptable salts thereof.

12. The method of claim 11, wherein the compound is selected from the group consisting of:
5-allylsulfanylmethyl-2,2,7,8-tetramethyl-chroman-6-ol;
2-(2-chloro-ethyl)-2,7,8-trimethyl-chroman-6-ol;
7-bromo-2,2,5,8-tetramethyl-chroman-6-ol;
2,2,5,8-tetramethyl-7-(3-methyl-butyl)-chroman-6-ol;
2,2-dimethyl-3,4-dihydro-2H-benzo[h]chromene-6,10-diol;
8-fluoro-2,2,5,7-tetramethylchroman-6-ol;
8-iodo-2,2,5,7-tetramethylchroman-6-ol;
2,5,7,8-tetramethyl-2-(thiophen-2-yl)chroman-6-ol;
8-isopentyl-2,2,5,7-tetramethylchroman-6-ol;
5-methoxy-2,2,7,8-tetramethylchroman-6-ol;
2,2,5,7-tetramethyl-8-(trifluoromethyl)chroman-6-ol;
5-(furan-2-yl)-2,2,7,8-tetramethylchroman-6-ol;
2,5,7,8-tetramethyl-2-styrylchroman-6-ol;
5-chloro-2,2,7,8-tetramethylchroman-6-ol; and
2-(3,5-difluoro-4-hydroxyphenyl)-2,5,7,8-tetramethylchroman-6-ol.

13. The method of claim 1, wherein the composition is coadministered with one or more diabetic medications.

14. The method of claim 13, wherein the diabetic medication is selected from the group consisting of pioglitizone, glimepiride, rosiglitazone, glipizide, metforministol, miglitol, repaglinide, acarbose, troglitazone, nateglinide, and combinations thereof.

15. A method of controlling blood glucose levels comprising administering to a subject with an elevated blood glucose level a composition comprising a pharmaceutically effective amount of one or more dual inhibitors that inhibit both 5-lipoxygenase (5-LO) and 12/15-lipoxygenase (12/15-LO) enzymes or both 5-lipoxygenase (5-LO) and 15-lipoxygenase (15-LO) enzymes.

16. The method of claim 15, wherein the subject is a mammal.

17. The method of claim 16, wherein the subject is a human.

18. The method of claim 15, wherein the dual inhibitor exhibits an in vitro IC₅₀ value of less than 5 micromolar in both a 5-lipoxygenase (5-LO) enzyme assay and a 12/15-lipoxygenase (12/15-LO) enzyme assay or in both a 5-lipoxygenase (5-LO) enzyme assay and a 15-lipoxygenase (15-LO) enzyme assay.

19. The method of claim 15, wherein the dual inhibitor is selected from the group consisting of nordihydroguaiaretic acid (NDGA), 8-fluoro-2,2,5,7-tetramethylchroman-6-ol, and 1-(4-hydroxyphenyl)-2,7,8-trimethyl-1,2,3,4-tetrahydroquinolin-6-ol.

20. The method of claim 19, wherein the dual inhibitor is 1-(4-hydroxyphenyl)-2,7,8-trimethyl-1,2,3,4-tetrahydroquinolin-6-ol.

21. The method of claim 15, wherein the dual inhibitor is a compound of Formula I: 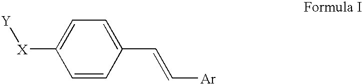
wherein,
Ar is an aryl group optionally substituted with one or more groups independently selected from alkyl, alkenyl, hydroxy, alkoxy, carboxy, amido, sulfonyl, aminosulfonyl, cyano, nitro and halogen;
X is a bond, an alkylene or an alkenylene group, and
Y is nitro, cyano, carboxy, amino, sulfonylamino, aminosulfonyl, alkylsulfonyl, arylsulfonyl, heterocyclylsulfonyl, or heterocyclic selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
or single stereoisomers and mixtures of stereoisomers, and pharmaceutically acceptable salts thereof.

22. The method of claim 15, wherein the dual inhibitor is a compound of Formula II: 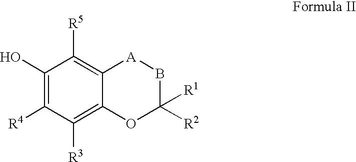
wherein,
R¹ is alkyl optionally substituted with halogen, hydroxy, cyano, amido or carboxy; or alkenyl optionally substituted with halogen, hydroxy, cyano, amido or carboxy;
R² is
alkyl optionally substituted with halogen, hydroxy, cyano, or carboxy;
alkenyl optionally substituted with halogen, hydroxy, cyano, or carboxy;
aryl optionally substituted with one or more groups independently selected from alkyl, alkenyl, hydroxy, alkoxy, carboxy, amido, sulfonyl, aminosulfonyl, oxo, cyano, nitro and halogen; or
heterocyclyl selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
R³ is hydrogen, optionally substituted alkyl, halogen, optionally substituted aryl or optionally substituted heterocyclyl;
R⁴ and R⁵ are independently of each other hydrogen, optionally substituted alkyl or halogen, or R⁴ and R⁵ may form an optionally substituted saturated, partially unsaturated or aromatic 5-6 membered ring optionally containing one or more heteroatoms or a C₇₋₁₂ bicyclo [a.b.c]alkyl ring where a, b, and c are 0 to 6, and
-A-B— is —CH₂—(CH₂)₀₋₂— or —CH═CH—;
or single stereoisomers, mixtures of stereoisomers, or pharmaceutically acceptable salts thereof.

23. A method of preventing insulinitis, comprising administering to a subject susceptible to insulinitis a composition comprising a pharmaceutically effective amount of one or more dual inhibitors of both 5-lipoxygenase (5-LO) and 12/15-lipoxygenase (12/15-LO) enzymes or both 5-lipoxygenase (5-LO) and 15-lipoxygenase (15-LO) enzymes.

24. The method of claim 23, wherein the subject is a mammal.

25. The method of claim 24, wherein the subject is a human.

26. The method of claim 23, wherein the dual inhibitor exhibits an in vitro IC₅₀ value of less than 5 micromolar in both a 5-lipoxygenase (5-LO) enzyme assay and a 12/15-lipoxygenase (12/15-LO) enzyme assay or in both a 5-lipoxygenase (5-LO) enzyme assay and a 15-lipoxygenase (15-LO) enzyme assay.

27. The method of claim 23, wherein the dual inhibitor is selected from the group consisting of nordihydroguaiaretic acid (NDGA), 8-fluoro-2,2,5,7-tetramethylchroman-6-ol, and 1-(4-hydroxyphenyl)-2,7,8-trimethyl-1,2,3,4-tetrahydroquinolin-6-ol.

28. The method of claim 27, wherein the dual inhibitor is 1-(4-hydroxyphenyl)-2,7,8-trimethyl-1,2,3,4-tetrahydroquinolin-6-ol.

29. The method of claim 23, wherein the dual inhibitor is a compound of Formula I: 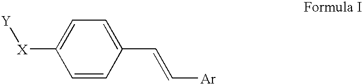
wherein,
Ar is an optionally substituted aryl group optionally substituted with one or more groups independently selected from alkyl, alkenyl, hydroxy, alkoxy, carboxy, amido, sulfonyl, aminosulfonyl, cyano, nitro and halogen;
X is a bond, an alkylene or an alkenylene group, and
Y is nitro, cyano, carboxy, amino (including cyclic amino), sulfonylamino, aminosulfonyl, alkylsulfonyl, arylsulfonyl, heterocyclylsulfonyl, or heterocyclic selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
or single stereoisomers and mixtures of stereoisomers, and pharmaceutically acceptable salts thereof.

30. The method of claim 23, wherein the dual inhibitor is a compound of Formula II: 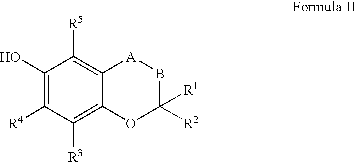
wherein,
R¹ is alkyl optionally substituted with halogen, hydroxy, cyano, amido or carboxy; or alkenyl optionally substituted with halogen, hydroxy, cyano, amido or carboxy;
R² is
alkyl optionally substituted with halogen, hydroxy, cyano, or carboxy;
alkenyl optionally substituted with halogen, hydroxy, cyano, or carboxy;
aryl optionally substituted with one or more groups independently selected from alkyl, alkenyl, hydroxy, alkoxy, carboxy, amido, sulfonyl, aminosulfonyl, oxo, cyano, nitro and halogen; or
heterocyclyl selected from morpholine, piperidine, piperazine, thiazole, thiazolidine, isothiazole, oxazole, isoxazole, pyrazole, pyrazolidine, pyrazoline, imidazole, imidazolidine, benzothiazole, pyridine, pyrazine, pyrimidine, pyridazine, pyrrole, pyrrolidine, quinoline, quinazoline, purine, carbazole, benzimidazole, thiophene, benzothiophene, pyran, tetrahydropyran, benzopyran, furan, tetrahydrofuran, indole, indoline, indazole, xanthene, thioxanthene, acridine, and quinuclidine, optionally substituted with one or more groups independently selected from alkyl, alkenyl, hydroxy, alkoxy, carboxy, amido, sulfonyl, aminosulfonyl, oxo, cyano, nitro and halogen;
R³ is hydrogen, optionally substituted alkyl, halogen, optionally substituted aryl or optionally substituted heterocyclyl;
R⁴ and R⁵ are independently of each other hydrogen, optionally substituted alkyl or halogen, or R⁴ and R⁵ may form an optionally substituted saturated, partially unsaturated or aromatic 5-6 membered ring optionally containing one or more heteroatoms or a C₇₋₁₂ bicyclo [a.b.c]alkyl ring where a, b, and c are 0 to 6, and
-A-B— is —CH₂—(CH₂)₀₋₂— or —CH═CH—;
or single stereoisomers and mixtures of stereoisomers, and pharmaceutically acceptable salts thereof.
